(19) United States
(12) Patent Application Publication
(10) Pub. No.: US 2007/0218142 A1
(43) Pub. Date: Sep. 20, 2007

(54) Pharmaceutical Formulations For The Prolonged Release Of Interleukins And Their Therapeutic Applications

(76) Inventors: Sophie Bignon, Lyon (FR); Remi Meyrueix, Lyon (FR); Olivier Soula, Meyzieu (FR)

Correspondence Address: PATTON BOGGS LLP, 8484 WESTPARK DRIVE, SUITE 900, MCLEAN, VA, 22102, US

(21) Appl. No.: 10/580,035

(22) PCT Filed: Nov. 19, 2004
(86) PCT No.: PCT/FR04/50607

§ 371 Date: Apr. 10, 2007

(30) Foreign Application Priority Data
Nov. 21, 2003 (FR) 0350888

(51) Int. Cl. A61K 9/14

(52) U.S. Cl. 424/499; 424/489

(57) ABSTRACT

The present invention relates to novel pharmaceutical formulations based on stable, fluid aqueous colloidal suspensions for the prolonged release of an interleukin (IL) (and one or more other possible active principles), and to the applications, especially therapeutic applications, of these formulations. The object of the invention is to propose a fluid pharmaceutical formulation for the prolonged release of interleukin(s) (and one or more other possible active principles) that makes it possible, after parenteral injection, significantly to increase the in vivo release time of the IL while at the same time reducing the plasma concentration peak of this IL, said formulation furthermore being stable on storage and also being biocompatible, biodegradable, non-toxic and non-immunogenic and having a good local tolerance. The formulation according to the invention is an aqueous colloidal suspension of low viscosity based on submicronic particles of water-soluble biodegradable polymer PO carrying hydrophobic groups (HG), said particles being non-covalently associated with at least one interleukin (and one or more other possible active principles) and forming a gelled deposit at the injection site, this gelling being caused by a protein present in the physiological medium.

The present invention relates to novel pharmaceutical formulations based on stable, fluid aqueous colloidal suspensions for the prolonged release of protein active principles, namely interleukins (IL), and to the therapeutic applications of these formulations. These active pharmaceutical formulations are of interest in both human and veterinary therapeutics.

Interleukins are a group of proteins belonging to the cytokine family. They have numerous activities which regulate the inflammatory response and the immunological response. However, their major role is activation and induction of the proliferation of T lymphocytes. IL-1, IL-2, IL-11 and IL-12 may be mentioned among the important members of this family. For example, IL-2 is produced by T lymphocytes activated by an antigen. The purpose of this IL-2 is to stimulate the other T lymphocytes in order to enable their activation and differentiation and thus to modulate the cell-mediated immune response.

Interleukins are used in therapeutics, but their well-known toxicity often remains the major cause of treatment interruption. For example, in the case of IL-2, the major events observed during the clinical use of IL-2 are fever, nausea, diarrhea, skin reactions, articular pains and apathy. In some cases this necessitates hospitalization for intensive care, and it is pointed out that, in these rare cases, the injection of IL-2 has been implicated in patient mortality.

Apart from the toxicity of IL, another factor to take into account in the prolonged release of the therapeutic proteins that interleukins represent is the need to ensure as far as possible that the patient's plasma protein concentration is close to the value observed in the healthy subject.

This objective is compromised by the short life of IL in the plasma; this makes it necessary to inject them repeatedly, which is very restricting. The plasma concentration of therapeutic protein then has a “sawtooth” profile characterized by high concentration peaks and very low concentration minima. The concentration peaks, which are very much greater than the basal concentration in the healthy subject, have very pronounced harmful effects due to the high toxicity of therapeutic proteins like interleukins, and more precisely the interleukin IL2. Furthermore, the concentration minima are below the concentration that is necessary to have a therapeutic effect, so the patient receives poor therapeutic cover and suffers serious long-term side effects.

Also, to ensure that the patient's plasma interleukin concentration is close to the ideal value for the treatment, the pharmaceutical formulation in question has to allow the prolonged release of the therapeutic protein so as to limit the variations in plasma concentration over time.

Furthermore, this active formulation should preferably meet the following specifications already familiar to those skilled in the art:

1-prolonged release of one or more active and non-denatured (unmodified) interleukins so that the plasma concentration is maintained at the therapeutic level,

2-liquid form sufficiently fluid to be easily injectable and sterilizable by filtration on filters with a pore size less than or equal to 0.2 micron,

3-stable liquid form,

4-biocompatibility and biodegradability,

5-atoxicity,

6-non-immunogenicity,

7-excellent local tolerance.

Several approaches have already been proposed in the prior art in an attempt to achieve these objectives.

In the first approach, the native therapeutic protein is modified by the covalent grafting of one or more polymer chains or by the covalent grafting of a protein such as human serum albumin (HSA). The protein modified in this way has a lower affinity for its receptors and its half-life in the general circulation increases considerably. The amplitude of the variation in concentration between the plasma protein concentration peaks and troughs is thereby considerably reduced. Thus, in its patent U.S. Pat. No. 4,766,106, Cetus proposes grafting a polyoxyethylene chain onto interleukin 2 in order to increase its solubility and life in the plasma. Likewise, Human Genome Science proposes (U.S. Pat. No. 5,876,969) covalently grafting interleukins onto human serum albumin in order to increase their life in the plasma. This step of chemical modification of the therapeutic protein generally has two major disadvantages. Firstly, the irreversible modification of the protein, which, now no longer being a human protein, can lead to toxicity and immunogenicity problems in the long term. The second disadvantage stems from the partial loss of bioactivity of the interleukin IL 2 modified in this way.

In a second approach, it has been proposed to increase the duration of action by using formulations containing at least one polymer and one active principle (“AP”) which are liquid at ambient temperature and in the ambient atmosphere, are injectable and become more viscous after injection, for example under the effect of a change in pH and/or temperature.

Thus, in this vein, patent U.S. Pat. No. 6,143,314 discloses an organic polymer solution for the controlled release of AP that forms a solid implant after injection. This solution comprises:
(A) 10 to 80% by weight of a base thermoplastic polymer which is biocompatible, biodegradable and insoluble in water or the physio-logical fluids (for example a polylactic and/or polyglycolic polymer);
(B) an organic solvent, such as N-methylpyrrolidone, which disperses in the physiological fluids;
(C) an active principle (AP);
(D) and finally 1 to 50% by weight of a controlled release agent consisting of a block copolymer of the polylactic-glycolic/polyethylene glycol type.
   After injection, (B) disperses or dissipates in the physiological fluids. (A) forms an implant that encapsulates (C), which is not covalently bonded to either (A) or (D) and is thus released slowly in vivo.

The main disadvantage of this technique is the use of an organic solvent (B), which is potentially denaturing for the AP (C) (e.g., therapeutic proteins) and toxic to the patient. In addition, in vivo hydrolysis of the polymer (A) generates an acid capable of causing problems of local tolerance.

PCT applications WO-A-99/18142 and WO-A-00/18821 relate to aqueous polymer solutions which contain an AP in dissolved or colloidal form, can be administered to warm-blooded animals, especially by injection, and form a gelled deposit of AP (e.g., insulin) in vivo because the physiological temperature is above their gelling point. The gel formed in this way releases the AP in a prolonged manner. These particular biodegradable polymers are ABA or BAB tri-blocks, where A=polylactic-glycolic copolymer (PLAGA) or polylactic polymer (PLA) and B=polyethylene glycol. The liquid =>gel transformation temperatures of these tri-block polymers are e.g., 36, 34, 30 and 26° C. Like the polymers (A) according to U.S. Pat. No. 6,143,314, in vivo hydrolysis of these ABA or BAB tri-block polymers produces acids which may not have the correct local tolerance.

PCT application WO-A-98/11874 describes pharmaceutical formulations comprising a lipophilic active principle, a gelling polymer (Gelrite®=deacetylated gellan gum or ethylhydroxy cellulose) and a surfactant. The polymer/surfactant interaction, and perhaps only the presence of electrolytes, such as Ca⁺⁺ ions, in a physiological concentration, in the case of the polymer Gelrite®, leads to the formation of a gel consisting of a polymer/surfactant aggregate, to which the lipophilic active principle bonds non-covalently. This formulation is intended for local administration to a target organ (e.g., the eye). The aggregate/active principle association which forms in situ allows the slow release of the active principle into the target organ.

A third approach adopted in an attempt to prolong the duration of action of a protein while preserving its bioactivity was to use a non-denatured therapeutic protein and incorporate it in microspheres or implants based on biocompatible polymers. This approach is illustrated especially by patent U.S. Pat. No. 6,500,448 and patent application US-A-2003/0133980, which describe a composition for the prolonged release of human growth hormone (hGH) in which the hormonal protein is first stabilized by complexation with a metal and then dispersed in a biocompatible polymer matrix. The biocompatible polymer is e.g., a polylactide, a polyglycolide or a poly(lactide-co-glycolide) copolymer. The composition is presented e.g., in the form of a suspension of microspheres in a solution of sodium carboxymethyl cellulose. This approach has several disadvantages: First of all, during the microsphere manufacturing process the protein is brought into contact with potentially denaturing organic solvents. Also, the microspheres are large (1 to 1000 microns), which is restricting in terms of injection and ease of sterilization on filters. Finally, problems of local tolerance can arise when the polymer is hydrolysed in situ.

According to a fourth approach, forms for the prolonged release of therapeutic proteins (especially interleukins) have been developed which consist of liquid suspensions of nanoparticles loaded with proteins. The latter have made it possible to administer the native protein in a liquid formulation of low viscosity.

According to a first method of prolonged release, the prolonged-release nanoparticle suspension consists of suspensions of liposomes in which the unmodified native therapeutic protein is encapsulated. After injection, the protein is released from the liposomes gradually, prolonging the time for which the protein is present in the general circulation. Thus, for example, in the article Cancer Res., 43, p. 546, 1983, Frossen et al. describe the encapsulation of antineoplastic agents in liposomes in order to enhance their therapeutic efficacy. The release of the drug, however, is too rapid to give a true prolonged release. In its patent U.S. Pat. No. 5,399,331, Liposome Company, Inc. proposes to improve the in vitro release time of interleukin 2 by grafting it covalently to the liposome, so said method suffers from the same shortcomings as the first “modified protein” approach referred to above.

To overcome the lack of stability of liposomes while at the same time retaining the advantages of a liquid nanoparticle formulation of low viscosity, Flamel Technologies has proposed a second method of prolonged release, in which the therapeutic protein is associated with nanoparticles of a water-soluble polymer that is “hydrophobically modified”, i.e., modified by the grafting of hydrophobic groups. This polymer is selected in particular from polyamino acids (polyglutamates or polyaspartates) carrying hydrophobic grafts.

One of the notable advantages of these hydrophobically modified polymers is that they spontaneously self-assemble in water to form nanoparticles.

Another advantage of these systems is that the therapeutic proteins or peptides associate spontaneously with the nanoparticles of hydrophobically modified polymers; this association is non-covalent and takes place without recourse either to a surfactant or to a potentially denaturing transformation process. It does not entail encapsulation of the protein in the microsphere, as disclosed in patent U.S. Pat. No. 6,500,448 and patent application US-A-2003/0133980. In total contrast, these nanoparticles of hydrophobically modified copolyamino acids spontaneously adsorb the proteins in solution without chemically modifying them or denaturing them and without subjecting them to aggressive treatment steps such as “emulsification” and “solvent evaporation”. The formulations can be stored in liquid or lyophilized form.

After injection, for example subcutaneously, these suspensions of nanoparticles loaded with proteins gradually release the bioactive non-denatured protein in vivo. Such non-covalent associations of protein active principle (AP)/poly[Glu] or poly[Asp] are disclosed in patent application WO-A-00/30618.

Said patent application particularly describes colloidal suspensions of pH 7.4 comprising associations of human insulin with nanoparticles of “hydrophobically modified” polyglutamate. The Table below shows the “hydrophobically modified” polyamino acids used and the degrees of association obtained in the Examples of WO-A-00/30618.

| | | Degree of |
|---|---|---|
| | | associ- |
| EXAM- | | ation |
| PLE | POLYMER | (%) |
| 1 | poly[(Glu-O—Na)_{0.63}-block-(Glu-O-methyl)_{0.37}] | 55 |
| 2 | poly[(Glu-O—Na)_{0.66}-block-(Glu-O-ethyl)_{0.34}] | 26 |
| 3 | poly[(Glu-O—Na)_{0.65}-block-(Glu-O-hexadecyl)_{0.35}] | 36 |
| 4 | poly[(Glu-O—Na)_{0.88}-block-(Glu-O-dodecyl)_{0.12}] | >90 |

These colloidal suspensions contain 1.4 mg/ml of insulin and 10 mg/ml of “hydrophobically modified” polyamino acid.

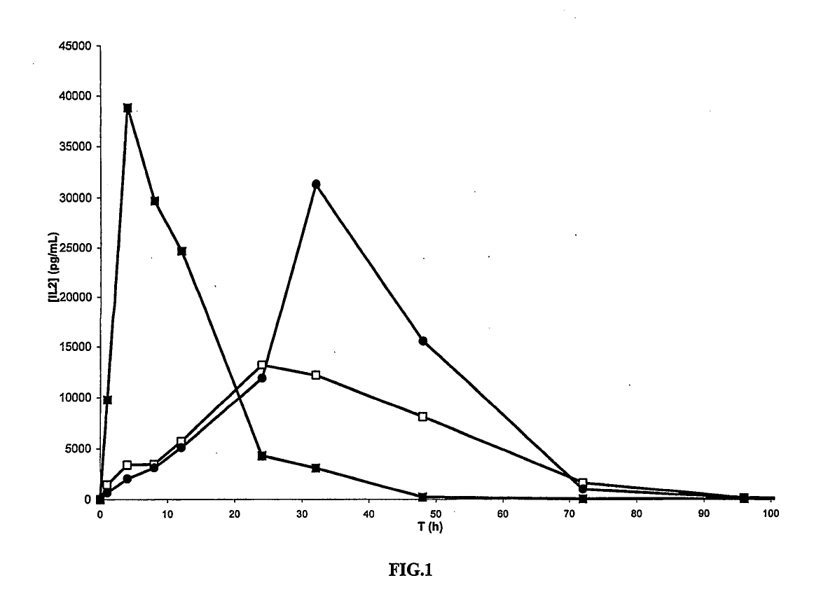

FIG. 1 of WO-A-00/30618 shows that the in vivo release time of the insulin vectorized by the above-mentioned suspensions is 12 h. This release time could profitably be increased.

Thus, even though said PCT application already represents a considerable advance, its technical content can be further optimized in respect of the specifications listed above, and especially as regards lengthening of the in vivo release time of interleukins.

Unpublished French patent applications no. 02 07008 of 07/06/2002, 02 09670 of 30/07/2002, 03 50190 of 28/05/2003 and 01 50641 of 03/10/2003 relate to novel water-soluble amphiphilic polyamino acids comprising aspartic units and/or glutamic units, in which at least some of these units carry hydrophobic grafts. Like the hydrophobically modified polyamino acids disclosed in patent application WO-A-00/30618, these novel polymer starting materials spontaneously form, in an aqueous liquid medium, colloidal suspensions of nanoparticles which can be used for the prolonged release of AP (insulin). They are biocompatible and biodegradable and proteins, particularly therapeutic proteins, adsorb spontaneously onto these nanoparticles without undergoing chemical modification or denaturation.

Said patent applications further relate to novel pharmaceutical, cosmetic, dietetic or phytosanitary compositions based on these polyamino acids.

The amphiphilic “hydrophobically modified” polyamino acids according to French patent application no. 02 07008 comprise aspartic units and/or glutamic units carrying hydrophobic grafts containing at least one alpha-tocopherol unit, e.g. polyglutamate or polyaspartate grafted with alpha-tocopherol of synthetic or natural origin.

Said unpublished patent application specifically discloses a colloidal suspension which contains nanoparticles formed of polymer/active protein associations and which is obtained by mixing 1 mg of a polyglutamate grafted with alpha-tocopherol and 7 mg of insulin in 1 ml of water at pH 7.0.

The amphiphilic “hydrophobically modified” polyamino acids according to French patent application no. 02 09670 comprise aspartic units and/or glutamic units carrying hydrophobic grafts that contain at least one hydrophobic unit and are joined to the aspartic and/or glutamic units via a rotating linkage containing two amide groups, and more precisely via a “spacer” of the lysine or ornithine type.

Said unpublished patent application specifically discloses a colloidal suspension which contains nanoparticles formed of polymer/active protein associations and which is obtained by mixing 10 mg of a polyglutamate grafted with palmitic acid via a lysine “spacer” and 200 IU of insulin (7.4 mg) in 1 ml of water at pH 7.4.

The amphiphilic “hydrophobically modified” polyamino acids according to French patent application no. 03 50190 comprise aspartic units and/or glutamic units, some of which carry at least one graft joined to an aspartic or glutamic unit via an “amino acid” “spacer” based on Leu and/or ILeu and/or Val and/or Phe, a C6-C30 hydrophobic group being joined to the “spacer” via an ester linkage.

Said unpublished patent application specifically discloses a colloidal suspension which contains nanoparticles formed of polymer/active protein associations and which is obtained by mixing an aqueous solution containing 10 mg of a polyglutamate grafted with a -Leu-OC8, -Val-OC12 or -Val-cholesteryl graft and 200 IU of insulin (7.4 mg) per millilitre of water at pH 7.4.

French patent application no. 01 50641 discloses anionic, amphiphilic linear homopolyamino acids comprising aspartic units or glutamic units, the ends of which carry hydrophobic groups containing from 8 to 30 carbon atoms.

In particular, the “hydrophobically modified” telechelic homopolyamino acids are e.g. a poly[GluONa] with PheOC18/C18 ends or a poly[GluONa] with PheOC18/alpha-tocopherol ends. Said unpublished patent application also describes a colloidal suspension which contains nanoparticles formed of polymer/active protein associations and which is obtained by mixing 10 mg of one of the above-mentioned polymers and 200 IU of insulin (7.4 mg) per millilitre of water at pH 7.4.

The in vivo release time of the insulin “vectorized” by the suspensions according to said unpublished patent applications could profitably be increased.

Whatever the case may be, none of this prior art relating to colloidal suspensions of nanoparticles of hydrophobically modified polyamino acids discloses a formulation that makes it possible to:
(I) sufficiently increase the release time of the active protein after parenteral injection, particularly subcutaneous injection;
(II) and/or reduce the plasma concentration peak of the active protein after injection of the formulation containing it.

Under these conditions, one of the important objectives of a present invention is therefore to propose a liquid pharmaceutical formulation for the prolonged release of IL which overcomes the deficiencies of the prior art and, in particular, makes it possible after parenteral (e.g. subcutaneous) injection to obtain a prolonged in vivo release time for non-denatured interleukins.

Another important objective of a present invention use a liquid pharmaceutical formulation for the prolonged release of interleukin(s) in vivo which is sufficiently fluid to be easily injectable and sterilizable by filtration on filters with a pore size less than or equal to 0.2 micron.

Another important objective of a present invention use a liquid pharmaceutical formulation for the prolonged release of interleukin(s) in vivo which is relatively stable on storage in both physicochemical and biological terms.

Another important objective of a present invention use a liquid pharmaceutical formulation for the prolonged release of interleukin(s) in vivo which has at least one of the following properties: biocompatibility, biodegradability, atoxicity, non-immunogenicity, good local tolerance.

Another important objective of a present invention use a pharmaceutical formulation for the slow prolonged release of interleukin(s) in vivo, this formulation being an aqueous colloidal suspension of low viscosity comprising submicronic particles of polymer PO that are auto-associated with at least one interleukin, the polymer PO being a water-soluble biodegradable polymer carrying hydrophobic groups.

Another important objective of a present invention use a pharmaceutical formulation for the slow prolonged release of interleukin(s) in vivo, this formulation being an aqueous colloidal suspension of low viscosity comprising submicronic particles of polymer PO that are auto-associated with at least one interleukin, the polymer PO being e.g. a polyamino acid formed of aspartic units and/or glutamic units, at least some of these units carrying grafts containing at least one hydrophobic group (HG), PO also being biodegradable, water-soluble and amphiphilic.

Another important objective of a present invention use derived products and/or precursors of the formulation referred to in the embodiments listed above.

It is particularly to the Applicant's credit to have developed aqueous liquid pharmaceutical formulations of low viscosity at the physiological temperature which, surprisingly, form a gelled deposit in vivo after easy parenteral administration to humans or warm-blooded mammals, the formation of this deposit not being triggered by a change in pH or temperature on parenteral injection, or by the dispersion of an organic solvent in the physiological medium. The gelled deposit formed in this way significantly increases the in vivo release time of the IL.

The invention thus relates to a liquid pharmaceutical formulation for the prolonged release of interleukin(s), this formulation comprising an aqueous colloidal suspension of low viscosity based on submicronic particles of water-soluble biodegradable polymer (PO) carrying hydrophobic groups (HG), said particles being non-covalently associated with at least one interleukin and optionally with at least one active principle (AP), characterized in that:
the dispersion medium of the suspension consists essentially of water,
said formulation is capable of being injected parenterally and then forming a gelled deposit in vivo, this formation of a gelled deposit:
   on the one hand being at least partly caused by at least one physiological protein present in vivo,
   and on the other hand making it possible to prolong and control the in vivo release time of the AP beyond 24 h after administration,
it is liquid under the injection conditions,
and it is also liquid at the physiological temperature and/or physiological pH and/or in the presence of:
   a physiological electrolyte in a physiological concentration,
   and/or at least one surfactant.

Advantageously, this gelling in vivo does not result from a change in pH and/or temperature or from the dispersion in vivo of one or more organic solvents that may be present in the injected formulation.

Without wishing to be bound by theory, one may consider that the physiological proteins present in vivo in physiological concentrations allow aggregation of the nanoparticles of PO associated with at least one interleukin. Such gelling takes place, e.g., in one hour or more, 24 h, 48 h or 72 h, inter alia.

In an optimized embodiment of the invention, the concentration of [PO] is such as to form a gelled deposit in vivo after parenteral injection.

According to one mode of definition, which is based not on an in vivo behaviour, as indicated above, but on an in vitro behaviour, the invention relates to a liquid pharmaceutical formulation for the prolonged release of interleukin(s) and optionally other active principle(s) (AP), this formulation:
being liquid in the ambient atmosphere,
also being liquid at the physiological temperature and/or physiological pH and/or in the presence of:
   a physiological electrolyte in a physiological concentration,
   and/or at least one surfactant,
and comprising an aqueous colloidal suspension of low viscosity based on submicronic particles of water-soluble biodegradable polymer PO carrying hydrophobic groups HG, said particles being non-covalently associated with at least one interleukin (and optionally at least one other active principle), and the dispersion medium of the suspension consisting essentially of water,
   characterized in that its concentration of [PO] is set at a sufficiently high value to allow the formation of a gelled deposit in vitro after parenteral injection, in the presence of at least one protein.

Preferably, the liquid pharmaceutical formulation according to the invention is characterized in that its concentration of [PO] is such that:
[PO]≧0.9.C1,
preferably 20.C1≧[PO]≧C1,
and particularly preferably 10.C1≧[PO]≧C1,
   where C1 is the “induced gelling” concentration of the particles of PO, as measured in an IG test.

The gelled deposit obtained after parenteral injection of the formulation allows a valuable prolongation of the release time of the protein, as well as a reduction in the plasma concentration peak of interleukin(s).

The release time of the interleukins is significantly increased compared with that of the formulations of the prior art, particularly those described in published PCT application WO-A-00/30618 and unpublished French patent applications no. 02 07008, 02 09670, 03 50190 and 01 50641.

The prolongation of the in vivo release time induced by the formulations according to the invention is all the more valuable because the interleukins released are still fully bioactive and non-denatured.

Interleukins in terms of the present disclosure are arbitrarily unmodified or modified interleukins, e.g. interleukins modified by the grafting of one or more polyoxyethylene groups. IL-1, IL-2, IL-11, IL-12 and IL-18 may be mentioned among the proteins of the interleukin family.

Throughout the present disclosure, the supramolecular arrangements of polymer PO associated or not associated with at least one interleukin and optionally with at least one other AP will be arbitrarily referred to as “submicronic particles” or “nanoparticles”. These correspond to particles with a mean hydrodynamic diameter (measured by the Md procedure defined below in the Examples) of, e.g., between 1 and 500 nm and preferably of between 5 and 250 nm.

Moreover, it is very important to note that these formulations are liquid, i.e., they advantageously have a very low viscosity, making them easy to inject. They only gel in vivo.

According to the invention, the qualifications “liquid”, “low viscosity” or “very low viscosity” advantageously correspond to a dynamic viscosity less than or equal to 5 Pa.s at 20° C. The reference measurement for the viscosity can be made e.g. at 20° C. using an AR1000 rheometer (TA Instruments) equipped with a cone-and-plate geometry (4 cm, 2°). The viscosity v is measured for a shear gradient of 10 s⁻¹.

Thus the viscosity of the formulations according to preferred embodiments of the invention can be, e.g., between 1.10⁻³ and 5 Pa.s, preferably between 1.10⁻³ and 0.8 Pa.s and particularly preferably between 1.10⁻³ and 0.5 Pa.s.

This low viscosity makes the formulations of the invention not only easy to inject parenterally, particularly intramuscularly or subcutaneously, inter alia, but also easy to sterilize at reduced cost by filtration on sterilization filters with a pore size of 0.2 μm.

This liquid state or low viscosity of the formulations of the invention exists both at injection temperatures corresponding to ambient temperatures, for example of between 4 and 30° C., and at the physiological temperature.

The formulation according to the invention is preferably an aqueous colloidal suspension of nanoparticles associated with one or more interleukins and optionally one or more AP. This means that, according to the invention, the dispersion medium of this suspension is formed primarily of water. In practice, this water represents e.g. at least 50% by weight, based on the total weight of the formulation.

In terms of the invention, the word “protein” denotes either a protein or a peptide, it being possible for this protein or peptide to be unmodified or modified, e.g., by the grafting of one or more polyoxyethylene groups.

“Physiological proteins” are understood in terms of the invention as meaning the endogenous proteins and/or peptides of warm-blooded mammals that are present at the injection site.

“Physiological temperature” is understood in terms of the invention as meaning the physiological temperature of warm-blooded mammals, e.g., about 37-42° C.

“Physiological pH” is understood in terms of the invention as meaning a pH, e.g., of between 6 and 7.6.

“Gel” is understood in terms of the invention as meaning a semisolid state into which the liquid formulation according to the invention is transformed spontaneously only by the presence of physiological protein(s), without the essential intervention of the physiological pH and/or the physiological temperature and/or the presence of a physiological electrolyte (e.g., Ca⁺⁺) and/or the dispersion (or dissipation) in vivo of an organic solvent that may be present in the injected formulation.

“Physiological electrolyte” is understood in terms of the invention as meaning any electrolyte species (for example Ca⁺⁺ ions) present in warm-blooded mammals.

“Physiological concentration” is understood in terms of the invention as meaning any physiological concentration encountered in warm-blooded mammals for the physiological medium in question.

In addition, the formulations according to the invention are non-toxic, have a good local tolerance and are stable.

It is also to the inventors' credit to have developed an in vitro IG test for selecting a population of preferred formulations according to the invention and determining the appropriate concentrations of PO in the formulations.

According to the invention, the IG test for measuring the gelling concentration C1 is a reference test for defining the critical concentration C1, hereafter called the induced gelling concentration C1, which characterizes each colloidal formulation according to the invention.

The IG test for determining the induced gelling concentration C1 is as follows:

The concentration C1 is determined by preparing colloidal formulations having variable concentrations of amphiphilic polymer according to the invention and a constant concentration of therapeutic protein. To this end, increasing amounts of dry powdered polymer are dissolved in deionized water. The solutions are kept at 25° C. for 16 hours, with magnetic stirring, before being mixed with a concentrated solution of therapeutic protein. The volume and concentration of this solution of therapeutic protein are adjusted to give the desired protein concentration for the formulation [for example 2.5 mg/ml of interleukin 2 (IL2)].

The colloidal formulations prepared in this way are mixed with a concentrated aqueous solution of bovine serum albumin (BSA) containing 30 mg/ml, and then centrifuged for 15 minutes at 3000 rpm. The mixtures are stirred gently for 24 h and then recovered for characterization.

The viscoelasticity measurements are made on a TA Instruments AR1000 rheometer equipped with a cone-and-plate geometry (diameter 4 cm and angle 1.59). A deformation of 0.01 rad, situated in the linear viscoelasticity domain, is imposed sinusoidally over a frequency range of between 0.1 and 300 rad/s. The temperature of the sample is kept constant at 20° C. by means of a Peltier cell.

The frequency spectra of the modulus of elasticity G′ and the modulus of viscosity or loss modulus G″ make it possible to define the characteristic relaxation time Tr, which is defined here as the reciprocal of the frequency at which the modulus of elasticity G′ intersects the modulus of viscosity G″. A detailed account of these questions will be found in the work by Ferry entitled Viscoelastic Properties of Polymers, J. D. Ferry, J. Wiley, N.Y., 1980, and in the article by J. REGALADO et al., Macromolecules, 1999, 32, 8580.

Measurement of the relaxation time Tr as a function of the polymer concentration of the formulation makes it possible to define the concentration C1 at which this time Tr exceeds 1 second. Examples of values of the gelling concentration C1 will be given in Example 6 below.

Likewise, it is possible to define the concentrations C0.1 and C10 at which the relaxation time exceeds 0.1 s and 10 s, respectively. These concentrations are classed in the following increasing order: C0.1<C1<C10.

In one variant of the formulation according to the invention:
[PO]≧C0.1,
preferably [PO]≧C1,
and particularly preferably [PO]≧C10.

According to an advantageous additional characteristic: [PO]≦20.C1.

In terms of the invention and throughout the present disclosure, the words “association” and “associate” employed to qualify the relationships between one or more active principles and the polymers PO (for example the polyamino acids) denote in particular that the active principle(s) is (are) bonded to the polymer(s) PO [for example the polyamino acid(s)] non-covalently, for example by electrostatic and/or hydrophobic interaction and/or hydrogen bonding and/or steric hindrance.

The polymers PO according to the invention are water-soluble biodegradable polymers carrying hydrophobic groups HG. The hydrophobic groups can be in reduced number relative to the rest of the chain and can be attached laterally to the chain or intercalated in the chain and be distributed randomly (random copolymer) or distributed in the form of sequences or grafts (block copolymers or sequenced copolymers).

Without implying a limitation, the hydrophobically modified polymers PO can be selected from the group comprising amphiphilic copolyamino acids, polysaccharides (preferably those in the subgroup comprising pullulans and/or chitosans and/or mucopolysaccharides), gelatins and mixtures thereof.

In one preferred embodiment of the invention, PO is selected from amphiphilic copolyamino acids.

In terms of the invention and throughout the present disclosure, the words “polyamino acid” cover both oligoamino acids comprising from 2 to 20 “amino acid” units and polyamino acids comprising more than 20 “amino acid” units.

Preferably, the polyamino acids according to the present invention are oligomers or homopolymers comprising glutamic or aspartic acid repeat units or copolymers comprising a mixture of these two types of “amino acid” units. The units in question in these polymers are amino acids having the D, L or D/L configuration and are bonded via their alpha or gamma positions in the case of the glutamate or glutamic unit and via their alpha or beta positions in the case of the aspartic or aspartate unit.

The preferred “amino acid” units of the main polyamino acid chain are those having the L configuration and a linkage of the alpha type.

In one particularly preferred embodiment of the invention, the polymer PO is a polyamino acid formed of aspartic units and/or glutamic units, at least some of these units carrying grafts containing at least one hydrophobic group HG. These polyamino acids are especially of the type described in PCT application WO-A-00/30618.

According to a first possibility, the PO of the formulation is (are) defined by general formula (I) below: 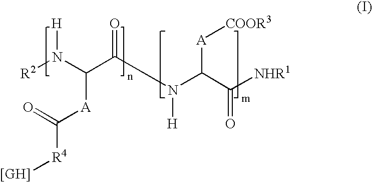
in which:
R¹ is H, a linear C2 to C10 alkyl or branched C3 to C10 alkyl, benzyl, a terminal amino acid unit or -R⁴-[HG];
R² is H, a linear C2 to C10 acyl or branched C3 to C10 acyl group, a pyroglutamate or -R⁴-[HG];
R³ is H or a cationic entity preferably selected from the group comprising:
   metal cations advantageously selected from the subgroup comprising sodium, potassium, calcium and magnesium,
   organic cations advantageously selected from the subgroup comprising:
      cations based on amine,
      cations based on oligoamine,
      cations based on polyamine (polyethylenimine being particularly preferred),
      cations based on amino acid(s) advantageously selected from the class comprising cations based on lysine or arginine,
   and cationic polyamino acids advantageously selected from the subgroup comprising polylysine and oligolysine;
R⁴ is a direct bond or a “spacer” based on 1 to 4 amino acid units;
A independently is a radical -CH₂-(aspartic unit) or -CH₂—CH₂-(glutamic unit);
n/(n+m) is defined as the molar grafting rate and its value is sufficiently low for PO, dissolved in water at pH 7 and at 25° C., to form a colloidal suspension of submicronic particles of PO, n/(n+m) preferably being between 1 and 25 mol % and particularly preferably between 1 and 15 mol %;
n+m is defined as the degree of polymerization and varies from 10 to 1000 and preferably between 50 and 300;
HG is a hydrophobic group.

According to a second possibility, the PO of the formulation has (have) one of general formulae (II), (III) and (IV) below: 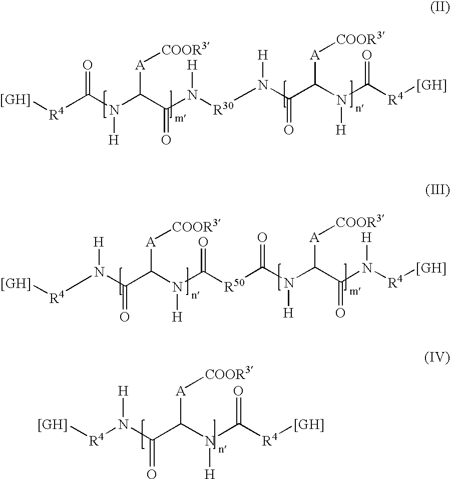
in which:
HG is a hydrophobic group;
R³⁰ is a linear C2 to C6 alkyl group;
R^{3′} is H or a cationic entity preferably selected from the group comprising:
   metal cations advantageously selected from the subgroup comprising sodium, potassium, calcium and magnesium,
   organic cations advantageously selected from the subgroup comprising:
      cations based on amine,
      cations based on oligoamine,
      cations based on polyamine (polyethylenimine being particularly preferred),
      cations based on amino acid(s) advantageously selected from the class comprising cations based on lysine or arginine,
   and cationic polyamino acids advantageously selected from the subgroup comprising polylysine and oligolysine;
R⁵⁰ is a C2 to C6 alkyl, dialkoxy or diamine group;
R⁴ is a direct bond or a “spacer” based on 1 to 4 amino acid units;
A independently is a radical -CH₂-(aspartic unit) or -CH₂—CH₂-(glutamic unit);
n′+m′ or n″ is defined as the degree of polymerization and varies from 10 to 1000 and preferably between 50 and 300.

Advantageously, the n HG groups of the PO each independently of one another are a monovalent radical of the formula below: 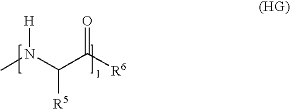
in which:
R⁵ is a methyl (alanine), isopropyl (valine), isobutyl (leucine), sec-butyl (isoleucine) or benzyl (phenylalanine);
R⁶ is a hydrophobic radical containing from 6 to 30 carbon atoms;
1 varies from 0 to 6.

According to one noteworthy characteristic of the invention, all or some of the hydrophobic groups R⁶ of the PO are independently selected from the group of radicals comprising:
a linear or branched alkoxy containing from 6 to 30 carbon atoms and optionally containing at least one heteroatom (preferably O and/or N and/or S) and/or at least one unit of unsaturation,
an alkoxy containing 6 to 30 carbon atoms, having one or more fused carbocyclic rings and optionally containing at least one unit of unsaturation and/or at least one heteroatom (preferably O and/or N and/or S),
an alkoxyaryl or an aryloxyalkyl having 7 to 30 carbon atoms and optionally containing at least one unit of unsaturation and/or at least one heteroatom (preferably O and/or N and/or S).

In practice and without implying a limitation, the hydrophobic radical R⁶ of the graft of the PO is derived from an alcohol precursor selected from the group comprising octanol, dodecanol, tetradecanol, hexadecanol, octadecanol, oleyl alcohol, tocopherol and cholesterol.

In a first embodiment of the invention, the main chains of the polyamino acids are alpha-L-glutamate or alpha-L-glutamic homopolymers.

In a second embodiment of the invention, the main chains of the polyamino acids are alpha-L-aspartate or alpha-L-aspartic homopolymers.

In a third embodiment of the invention, the main chains of the polyamino acids are alpha-L-aspartate/alpha-L-glutamate or alpha-L-aspartic/alpha-L-glutamic copolymers.

Advantageously, the distribution of the aspartic and/or glutamic units of the main polyamino acid chain of the PO is such that the resulting polymer is either random or of the block type or of the multiblock type.

Preferably, the PO used in the formulation according to the invention has a molecular weight of between 2000 and 100,000 g/mol and preferably of between 5000 and 40,000 g/mol.

In a first preferred embodiment of the formulation, the hydrophobic radical R⁶ of the graft of the PO is derived from an alcohol precursor formed of tocopherol:
1%≦[n/(n+m)]×100≦10%,
preferably 3.5%≦[n/(n+m)]×100≦7.5%,
n+m varies from 100 to 400 and preferably between 120 and 300.

In a second preferred embodiment of the formulation, the hydrophobic radical R⁶ of the graft of the PO is derived from an alcohol precursor formed of cholesterol:
1%≦[n/(n+m)]×100≦10%,
preferably 3.5%≦[n/(n+m)]×100≦6.5%,
n+m varies from 100 to 400 and preferably between 120 and 300.

In both these preferred embodiments of the formulation of the invention, the concentration of polymer [PO] is advantageously between 15 and 50 mg/ml.

In one variant, the PO of the formulation according to the invention carries at least one graft of the polyalkylene glycol type bonded to a glutamate and/or aspartate unit.

Advantageously, this graft of the polyalkylene glycol type has formula (V) below: 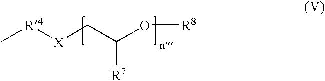
in which:
R⁴ is a direct bond or a “spacer” based on 1 to 4 amino acid units;
X is a heteroatom selected from the group comprising oxygen, nitrogen and sulfur;
R⁷ and R⁸ independently are H or a linear C1 to C4 alkyl;
n′″ varies from 10 to 1000 and preferably from 50 to 300.

In practice, the polyalkylene glycol is e.g. a polyethylene glycol.

It is desirable according to the invention for the molar percentage of polyalkylene glycol grafting to vary from 1 to 30%.

The polyamino acids PO are also extremely valuable in that, with an adjustable grafting rate, they disperse in water at pH 7.4 (e.g., with a phosphate buffer) to give colloidal suspensions.

Furthermore, the interferon active principles or other AP selected from proteins, peptides and small molecules can associate spontaneously with nanoparticles comprising these polyamino acids PO.

It should be understood that the PO based on polyamino acids contain carboxyl groups which are either neutral (COOH form) or ionized (COO⁻ anion), depending on the pH and the composition. For this reason, the solubility in an aqueous phase is a direct function of the proportion of free COOH groups in the PO (not grafted with the hydrophobic unit) and of the pH. In aqueous solution the countercation can be a metal cation such as sodium, calcium or magnesium, or an organic cation such as triethanolamine, tris(hydroxymethyl)aminomethane or a polyamine like polyethylenimine.

The PO of the polyamino acid type that are capable of being used in the formulation of the invention are obtained e.g. by methods known to those skilled in the art. Random polyamino acids can be obtained by grafting the hydrophobic graft, previously functionalized with the “spacer”, directly onto the polymer by a conventional coupling reaction. Block or multiblock polyamino acids PO can be obtained by sequential polymerization of the corresponding amino acid N-carboxy anhydrides (NCA).

For example, a homopolyglutamate or homopolyaspartate polyamino acid or a block, multiblock or random glutamate/aspartate copolymer is prepared by conventional methods.

To obtain a polyamino acid of the alpha type, the most common technique is based on the polymerization of amino acid N-carboxy anhydrides (NCA), which is described, e.g., in the article “Biopolymers”, 1976, 15, 1869, and in the work by H. R. Kricheldorf entitled “Alpha-amino acid N-carboxy anhydrides and related heterocycles”, Springer Verlag (1987). The NCA derivatives are preferably NCA-O-Me, NCA-O-Et or NCA-O-Bz derivatives (Me=methyl, Et=ethyl and Bz=benzyl). The polymers are then hydrolysed under appropriate conditions to give the polymer in its acid form. These methods are based on the description given in patent FR-A-2 801 226 to the Applicant. A number of polymers that can be used according to the invention, for example of the poly(alpha-L-aspartic), poly(alpha-L-glutamic), poly(alpha-D-glutamic) and poly(gamma-L-glutamic) types of variable molecular weights, are commercially available. The polyaspartic polymer of the alpha-beta type is obtained by the condensation of aspartic acid (to give a polysuccinimide) followed by basic hydrolysis (cf. Tomida et al., Polymer, 1997, 38, 4733-36).

Coupling of the graft with an acid group of the polymer is easily effected by reacting the polyamino acid in the presence of a carbodiimide as coupling agent, and optionally a catalyst such as 4-dimethylaminopyridine, in an appropriate solvent such as dimethylformamide (DMF), N-methylpyrrolidone (NMP) or dimethyl sulfoxide (DMSO). The carbodiimide is e.g., dicyclohexylcarbodiimide or diisopropylcarbodiimide. The grafting rate is controlled chemically by the stoichiometry of the constituents and reactants or by the reaction time. The hydrophobic grafts functionalized with a “spacer” are obtained by conventional peptide coupling or by direct condensation under acid catalysis. These techniques are well known to those skilled in the art.

A block or multiblock copolymer is synthesized using NCA derivatives previously synthesized with the hydrophobic graft. For example, the hydrophobic NCA derivative is copolymerized with NCA-O-benzyl and the benzyl groups are then selectively removed by hydrolysis.

The synthesis of polyamino acids PO preferably produces aqueous suspensions of nanoparticles of PO.

Such suspensions can be converted to powdered nanoparticles of PO by drying in an appropriate manner known to those skilled in the art, for example by heating (oven, etc.), evacuation, use of desiccants, lyophilization or atomization.

These nanoparticles of PO, in suspension or in the pulverulent state, form a starting material for the preparation of the formulations according to the invention.

It may be stated at this point that the formulations according to the invention result from the non-covalent association of nanoparticles based on at least one PO and at least one AP, in an aqueous liquid medium.

For the preparation, the PO and/or the interleukin(s) (and/or any additional AP) can be in solid form (preferably a powder) and/or in liquid form (preferably a colloidal aqueous suspension).

In terms of the present disclosure, interleukin(s)/PO association means that the interleukin(s) is (are) associated with the polymer(s) PO [e.g., one or more polyamino acids] by one or more bonds other than a covalent chemical bond or covalent chemical bonds.

The techniques for associating one or more interleukins with the PO according to the invention are described in particular in patent application WO-A-00/30618. They consist in incorporating at least one interleukin (and one or more other possible active principles) into the liquid medium containing nanoparticles of PO to give a colloidal suspension of nanoparticles loaded or associated with one or more interleukins (and one or more other possible active principles).

The invention therefore further relates to a process for the preparation of the above-mentioned formulation.

In a first preferred mode of carrying out the invention, this process is characterized in that it consists essentially in:
taking a colloidal suspension of nanoparticles of at least one PO,
mixing this colloidal suspension of nanoparticles of PO with at least one interleukin (and one or more other possible active principles), preferably in aqueous solution,
optionally adding at least one excipient,
adjusting the pH and/or the osmolarity if necessary, and
optionally filtering the resulting suspension.

Advantageously, the interleukin(s) (and one or more other possible active principles) is (are) in the form of an aqueous suspension or solution for mixing with the colloidal suspension of nanoparticles of PO.

In a second mode of carrying out the invention, this process is characterized in that it consists essentially in:
taking a powder of at least one polymer PO,
mixing this powder with an aqueous suspension or solution of at least one interleukin (and one or more other possible active principles), preferably in aqueous solution,
optionally adding at least one excipient,
adjusting the pH and/or the osmolarity if necessary, and
optionally filtering the resulting suspension.

The formulations obtained in this way can also be converted to gels, powder or film by the conventional methods known to those skilled in the art, such as concentration by diafiltration or evaporation, coating, atomization or lyophilization, inter alia. These methods can optionally be combined.

Hence there is a third mode of carrying out the process for the preparation of liquid formulations according to the invention, this third mode consisting essentially in:
taking a powder produced by drying the liquid formulation according to the invention as defined above,
mixing this powder with an aqueous liquid medium, preferably with stirring,
optionally adding at least one excipient,
adjusting the pH and/or the osmolarity if necessary, and
optionally filtering the resulting suspension.

Examples of excipients that can be added are antimicrobial agents, buffers, antioxidants and agents for adjusting the isotonicity, which are known to those skilled in the art. Reference may be made, e.g., to the work entitled Injectable Drug Development, P. K. Gupta et al., Interpharm Press, Denver, Colo., 1999.

If appropriate, the liquid formulation can be sterilized by filtration on filters with a porosity of 0.2 μm, for example. It can then be injected directly into a patient.

All these examples of the preparation of liquid formulations according to the invention are advantageously carried out in the ambient atmosphere and at ambient temperature (e.g., 25° C.).

In one valuable variant of the formulation according to the invention, its weight fraction of interleukin(s) not associated with the submicronic particles [non-associated interleukin(s)], in %, is such that:
[non-associated interleukin(s)]≦1,
preferably [non-associated interleukin(s)]≦0.5,
most preferably [non-associated interleukin(s)]≦0.1.

According to the invention, the preferred interleukin is interleukin 2.

According to another of its features, the invention encompasses any derived product obtained from the liquid formulation according to the invention as defined above, and comprising submicronic particles formed of PO/interleukin non-covalent associations as defined above.

In practice, these derived products can consist especially of powders, gels, implants or films, inter alia.

The invention further relates to any precursor of the injectable liquid formulation as defined above.

Still on the subject of these derived products, it must be emphasized that the invention further relates to a process for the preparation of a powder derived from the formulation as defined above, this process being characterized in that said powder is obtained by drying the formulation as defined above.

The formulation according to the invention is preferably pharmaceutical without excluding cosmetic, dietetic or phytosanitary formulations comprising at least one PO as defined above, at least one interleukin and optionally at least one other active principle.

According to the invention, the possible additional active principle other than an interleukin can be a protein, a glycoprotein, a protein bonded to one or more polyalkylene glycol chains [preferably polyethylene glycol (PEG) chains: “PEGylated protein”], a polysaccharide, a liposaccharide, an oligonucleotide, a polynucleotide or a peptide.

This additional active principle can be selected from haemoglobins, cytochromes, albumins, interferons, cytokines, antigens, antibodies, erythropoietin, insulin, growth hormones, factors VIII and IX, haemopoiesis stimulating factors, and mixtures thereof.

In one variant, this additional active principle is a “small” hydrophobic, hydrophilic or amphiphilic organic molecule, examples being peptides such as leuprolide or cyclosporin, or small molecules such as those belonging to the anthracycline, taxoid or camptothecin families, and mixtures thereof.

The primary properties of the formulation according to the invention include its injectability and its ability to form a deposit at the injection site, in vivo, by gelling or by aggregation of the nanoparticles, in the presence of physiological proteins or analogues.

The formulation according to the invention can be injected especially by the parenteral, subcutaneous, intramuscular, intradermal, intraperitoneal or intracerebral route or into a tumour.

The formulation according to the invention can also be administered by the oral, nasal, vaginal, ocular or buccal route.

Advantageously, the formulation is intended for the preparation of drugs, particularly for administration by the parenteral, subcutaneous, intramuscular, intradermal, intraperitoneal or intracerebral route or into a tumour, or by the oral, nasal, vaginal or ocular route.

Although the formulation according to the invention is preferably pharmaceutical, this does not exclude cosmetic, dietetic or phytosanitary formulations comprising at least one PO as defined above and at least one corresponding active principle.

According to yet another of its features, the invention relates to a process for the preparation of drugs, particularly for administration by the parenteral, subcutaneous, intramuscular, intradermal, intraperitoneal or intracerebral route or into a tumour, or by the oral, nasal, vaginal or ocular route, characterized in that it consists essentially in using at least one formulation defined above and/or any derived product and/or any precursor of said formulation.

The invention further relates to a method of therapeutic treatment consisting essentially in administering the formulation as described in the present disclosure by the parenteral, subcutaneous, intramuscular, intradermal, intraperitoneal or intracerebral route or into a tumour, or by the oral, nasal, vaginal or ocular route.

In one particular variant of the invention, this method of therapeutic treatment consists essentially in administering the formulation as described above by injection by the parenteral, subcutaneous, intramuscular, intradermal, intraperitoneal or intracerebral route or into a tumour, preferably in such a way that it forms a gelled/crosslinked deposit at the injection site.

The invention will be understood more clearly and its advantages and variants will become clearly apparent from the Examples below, which describe the synthesis of the PO formed of polyamino acids grafted with a hydrophobic group, and their conversion to a system for the prolonged release of an interleukin, namely a formulation according to the invention (stable aqueous colloidal suspension), and demonstrate the ability of such a system not only to associate with an interleukin, but also, in particular, to gel/crosslink in order to release the interleukins in a very prolonged manner in vivo.

### DESCRIPTION OF THE FIGURES

FIG. 1: Curves of the plasma IL2 concentrations (picograms/mi) recorded in the monkey after subcutaneous injection of:
the IL2 formulation (E) according to the invention (Example 7):→curve -□-□-,
the control IL2 formulation (F) not according to the invention (Example 7):→curve -●-●-,
and the control IL2 formulation (G) not according to the invention (Example 7):→curve -▪-▪-,
   as a function of the time T in hours and at an IL2 dose of 0.5 mg/kg.

### EXAMPLES

### Example 1

Amphiphilic Polymer P1

Synthesis of a Polyglutamate Grafted With Alpha-tocopherol of Synthetic Origin

5.5 g of an alpha-L-polyglutamate (having a molecular weight equivalent to about 10,000 Da, relative to a polyoxyethylene standard, and obtained by the polymerization of NCAGluOMe followed by hydrolysis, as described in patent application FR-A-2 801 226) are solubilized in 92 ml of dimethylformamide (DMF) by heating at 40° C. for 2 hours. Once the polymer is solubilized, the temperature is allowed to drop to 25° C. and 1.49 g of D,L-alpha-tocopherol (>98%, obtained from Fluka®), previously solubilized in 6 ml of DMF, 0.09 g of 4-dimethylaminopyridine, previously solubilized in 6 ml of DMF, and 0.57 g of diisopropylcarbodiimide, previously solubilized in 6 ml of DMF, are added in succession. After 8 hours at 25° C., with stirring, the reaction medium is poured into 800 ml of water containing 15% of sodium chloride and hydrochloric acid (pH 2). The precipitated polymer is then recovered by filtration and washed with 0.1 N hydrochloric acid and then with water. The polymer is subsequently resolubilized in 75 ml of DMF and then reprecipitated in water containing, as previously, salt and acid to pH 2. After two washes with water, the precipitate is washed several times with diisopropyl ether. The polymer is then dried in an oven under vacuum at 40° C. to give a yield in the order of 85%.

### Example 2

### Amphiphilic Polymers P1, P2, P3, P4, P5 and P6

These polymers are obtained in the same way as the polymer P1. Table 1 below summarizes the characteristics of these polymers. Those of the polymer P1 are given by way of comparison.

TABLE 1

| | Mn¹ g/mol | | | |
|---|---|---|---|---|
| | of the | Hydrophobic | % grafting | Mn¹ g/mol of |
| Polymer | polyglutamate | group | (NMR)² | the polymer |
| P1 | 10,000 | alpha-tocopherol³ | 7 | 13,900 |
| P2 | 10,000 | alpha-tocopherol³ | 4 | 14,400 |
| P3 | 16,900 | alpha-tocopherol³ | 4 | 15,200 |
| P4 | 10,000 | cholesterol | 5 | 11,500 |
| P5 | 16,900 | cholesterol | 5 | 12,900 |
| P6 | 10,000 | n-dodecanol | 15 | 11,500 |

¹in polyoxyethylene equivalents
²molar grafting rate estimated by proton NMR
³of synthetic origin

### Example 3

### Preparation of a Long-Acting Interleukin 2 (IL2) Formulation According to the Present Invention, Based on the Polymer P3

Lyophilized powder of the amphiphilic polymer and sterile water are introduced into a flask in the amount necessary to give a polymer concentration X=1.3 times the desired final concentration in the formulation. Dissolution of the polymer is continued for 16 hours, with magnetic stirring.

The necessary amount of lyophilized L2 (Prospec) is concentrated to X/(X-1) times the desired final concentration.

The precise concentration of the concentrated IL2 solution is determined by UV assay at 280 nm using a Perkin Elmer Lambda 35 UV spectrophotometer.

This L2 solution is filtered on a 0.8-0.2 μm filter and stored at 4° C. Its pH is adjusted to 11 by adding 1 M NaOH. The ratio of the protein concentration of this solution to the desired concentration in the formulation is called Y.

The protein solution and the polymer solution are then mixed at ambient temperature. X-1 volumes of protein solution are added per volume of polymer. The pH and the osmolarity are adjusted to 7.4±0.2 and 300±20 mOsm, respectively.

Thus, to prepare a long-acting IL2 formulation according to the invention, based on the polymer P3, containing 20 mg/ml of polymer P3 and 2.5 mg/ml of IL2, the initial polymer solution is concentrated to 26 mg/ml. The initial IL2 solution is concentrated to 11 mg/ml. 0.3 volume of protein solution is added per volume of polymer.

### Example 4

### Measurement of the Mean Hydrodynamic Diameter of the Nanoparticles of Different Polymers PO According to the Invention

The mean hydrodynamic diameter of the particles of polymer PO according to the invention is measured by the Md procedure defined below.

The PO solutions are prepared at concentrations of 1 or 2 mg/ml in 0.15 M NaCl medium and stirred for 24 h. These solutions are then filtered on a 0.8-0.2 μm filter before being analysed by dynamic light scattering using a Brookhaven apparatus operating with a vertically polarized laser beam of wavelength 488 nm. The hydrodynamic diameter of the nanoparticles of polymer PO is calculated from the electric field autocorrelation function by the summation method, as described in the work “Surfactant Science Series” volume 22, Surfactant Solutions, Ed. R. Zana, chap. 3, M. Dekker, 1984.

The following results are obtained for the polymers PO P2, P3, P4 and P6 of Example 2:

TABLE 2

| Polymer | Mean hydrodynamic diameter (nm) |
|---|---|
| P2 | 60 |
| P3 | 90 |
| P4 | 30 |
| P6 | 15 |

### Example 5

Spontaneous Association of a Protein With the Nanoparticles of Polymer PO

A 25 mM phosphate buffer solution is prepared from powdered NaH₂PO₄ (Sigma ref. S-075 1) and adjusted to pH 7.2 with 1 N sodium hydroxide solution (SDS ref. 3470015).

A colloidal suspension of nanoparticles of polymer P1 is prepared by dissolving 5 mg/ml of the lyophilized polymer overnight in the above phosphate buffer solution.

A stock solution of BSA (Sigma A-2934) is prepared by dissolving 10 mg/ml of the protein for two hours in the same buffer.

The stock solutions and the buffer are filtered on a 0.22 μm filter.

Mixtures are made up by the addition of predetermined volumes of the two stock solutions and dilution in the phosphate buffer, ultimately giving a range of samples having a constant polymer concentration (0.1 mg/ml) and increasing protein concentrations (0 to 1.8 mg/ml).

The samples are left to associate for 5 hours at 25° C., after which they are analysed by capillary electrophoresis using a so-called frontal method, which allows the protein and the protein-polymer complex to be visualized separately. Further details on this technique may be obtained by consulting the following article: Gao J. Y., Dublin P. L., Muhoberac B. B., Anal. Chem., 1997, 69, 2945. The analyses are performed on an Agilent G16000A apparatus equipped with a fused silica bubble capillary (type G1600-62-232). The height of the first plateau, corresponding to the free protein, makes it possible to determine the concentration of non-associated BSA. Experience shows that, for amounts of proteins below 0.1 g of protein per g of polymer, the protein is associated with the nanoparticles of polymer.

### Example 6

Determination of the Gelling Concentration C1 for the Polymers PO P1, P3 and P6

The IG test is applied to formulations of IL2 associated with the polymers P1, P3 and P6 of Examples 1 and 2. The protein concentrations of these formulations are shown in the Table below. The relaxation time of the formulations in the presence of BSA (concentration 30 mg/ml) is measured by the procedure of the IG test. The critical concentration C1, for which the relaxation time exceeds 1 s, is shown in Table 3 for IL2.

TABLE 3
Induced gelling concentration for IL2 formulations

| | Polymer | | |
|---|---|---|---|
| | P1 | P3 | P6 |
| IL2 concentration | 2.5 | 2.5 | 2.5 |
| (mg/ml) | | | |
| Concentration C1 | 17 | 17 | >50 |
| (mg/ml) | | | |

### Example 7

Pharmacokinetics of Interleukin 2 (IL2) in the Monkey After Subcutaneous Injection of Various Formulations Based on Amphiphilic Polyamino Acids

The following formulations are prepared by the procedure described in Example 3:

TABLE 4

| | | Polymer | IL2 |
|---|---|---|---|
| | | concentration | concentration |
| Reference | Polymer | (mg/ml) | (mg/ml) |
| E | P1 | 30 | 2.5 |
| F | P3 | 20 | 2.5 |
| G | P6 | 40 | 2.5 |

The formulations E and F, whose polymer concentrations are greater than the gelling concentration C1 measured in Example 6, therefore belong to the selection according to the invention. On the other hand, the concentration of the formulation G is less than the gelling concentration C1, so said formulation does not belong to the selection according to the invention.
These formulations are injected into Cynomolgus monkeys at a dose of 0.5 mg/kg. Plasma samples are taken at 1, 5, 11, 24, 36, 48, 72, 96, 120, 144, 168 and 240 hours. The plasma IL2 concentration is measured on these samples, by ELISA (Immunotech IM 3583 kit).

The times Tmax and T50 for the formulations E, F and G are shown in Table 5 below.

TABLE 5

| Reference of | | |
|---|---|---|
| formulation | Tmax (h) | T50 (h) |
| E | 32 | 34.5 |
| F | 32 | 37.5 |
| G | 4 | 10.5 |

Thus the formulations E and F, which belong to the selection according to the invention, have a considerably longer release time than the formulation G, which does not belong to the selection according to the invention.

### Example 8

Observation of the Gelling of the Formulations According to the Invention In Vivo After Subcutaneous Injection

The subcutaneous behaviour of the formulations according to the invention was studied in the domestic pig. Six domestic pigs were injected under the abdominal skin, to a depth of 4 mm, with 0.3 ml of the following formulations:

Formulation A: isotonic aqueous solution, pH 7.3, of the polymer P6 of Example 2 at a concentration of 45 mg/ml.

Formulation B: isotonic aqueous solution, pH 7.3, of the polymer P1 of Example 1 at a concentration of 20 mg/ml.

Samples were taken from the injected sites 72 hours after administration. Histological examination discloses the presence of a gelled deposit of polymer for the formulation B. It takes the form of uniformly coloured plaques. By contrast, this phenomenon is not observed for the formulation A, for which the polymer has infiltrated between the collagen fibres.

It may be emphasized that the polymer matrix B is perfectly biodegradable since the tissue has completely returned to its normal state after 21 days.

1. Liquid pharmaceutical formulation for the prolonged release of interleukin(s), this formulation comprising an aqueous colloidal suspension of low viscosity based on submicronic particles of water-soluble biodegradable polymer (PO) carrying hydrophobic groups (HG), said particles being non-covalently associated with at least one interleukin and optionally with at least one other active principle active principle (AP), characterized in that:
the dispersion medium of the suspension consists essentially of water,
said formulation is capable of being injected parenterally and then forming a gelled deposit in vivo, this formation of a gelled deposit:
on the one hand being at least partly caused by at least one physiological protein present in vivo,
and on the other hand making it possible to prolong and control the in vivo release time of the AP beyond 24 h after administration,
it is liquid under the injection conditions,
and it is also liquid at the physiological temperature and/or physiological pH and/or in the presence of:
a physiological electrolyte in a physiological concentration,
and/or at least one surfactant.

2. Formulation according to claim 1, characterized in that its concentration of [PO] is set at a sufficiently high value to allow the formation of a gelled deposit in vivo after parenteral injection, in the presence of at least one physiological protein.

3. Liquid pharmaceutical formulation for the prolonged release of interleukin(s) and optionally other active principle(s) (AP), this formulation:
being liquid in the ambient atmosphere,
also being liquid at the physiological temperature and/or physiological pH and/or in the presence of:
a physiological electrolyte in a physiological concentration,
and/or at least one surfactant,
and comprising an aqueous colloidal suspension of low viscosity based on submicronic particles of water-soluble biodegradable polymer PO carrying hydrophobic groups HG, said particles being non-covalently associated with at least one active principle AP, and the dispersion medium of the suspension consisting essentially of water,
characterized in that its concentration of [PO] is set at a sufficiently high value to allow the formation of a gelled deposit in vitro after parenteral injection, in the presence of at least one protein.

4. Formulation according to any one of the preceding claims, characterized in that its concentration of [PO] is such that:
[PO]≧0.9.C1,
preferably 20.C1≧[PO]≧Cl,
and particularly preferably 10.C1≧[PO]≧Cl,
where C1 is the “induced gelling” concentration of the particles of PO, as measured in an IG test.

5. Formulation according to any one of the preceding claims, characterized in that its viscosity is less than or equal to 5 Pa.s at 25° C.

6. Formulation according to any one of the preceding claims, characterized in that the polymer PO is a polyamino acid formed of aspartic units and/or glutamic units, at least some of these units carrying grafts containing at least one hydrophobic group (HG).

7. Formulation according to claim 6, characterized in that the PO is (are) defined by general formula (I) below: 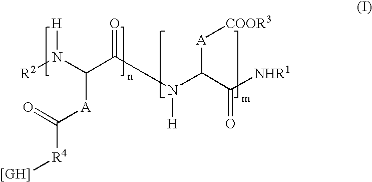
in which:
R¹ is H, a linear C2 to C10 alkyl or branched C3 to C10 alkyl, benzyl, a terminal amino acid unit or -R⁴-[HG];
R² is H, a linear C2 to C10 acyl or branched C3 to C10 acyl group, a pyroglutamate or -R⁴-[HG];
R³ is H or a cationic entity preferably selected from the group comprising:
metal cations advantageously selected from the subgroup comprising sodium, potassium, calcium and magnesium,
organic cations advantageously selected from the subgroup comprising:
cations based on amine,
cations based on oligoamine,
cations based on polyamine (polyethylenimine being particularly preferred),
cations based on amino acid(s) advantageously selected from the class comprising cations based on lysine or arginine,
and cationic polyamino acids advantageously selected from the subgroup comprising polylysine and oligolysine;
R⁴ is a direct bond or a “spacer” based on 1 to 4 amino acid units;
A independently is a radical -CH₂-(aspartic unit) or -CH₂—CH₂-(glutamic unit);
n/(n+m) is defined as the molar grafting rate and varies from 0.5 to 100 mol %;
n/(n+m) is defined as the molar grafting rate and its value is sufficiently low for PO, dissolved in water at pH 7 and at 25° C., to form a colloidal suspension of submicronic particles of PO, n/(n+m) preferably being between 1 and 25 mol % and particularly preferably between 1 and 15 mol %;
n+m varies from 10 to 1000 and preferably between 50 and 300;
HG is a hydrophobic group.

8. Formulation according to claim 6, characterized in that the PO has (have) one of general formulae (II), (III) and (IV) below: 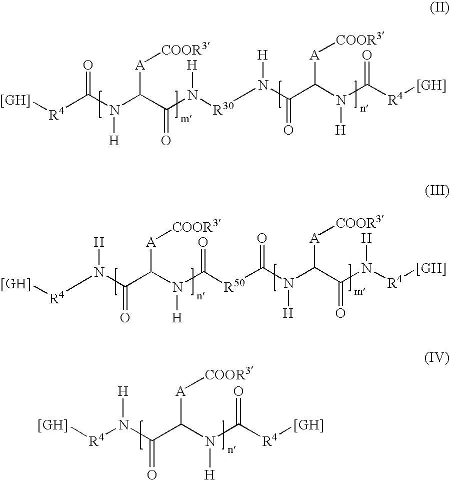
in which:
HG is a hydrophobic group;
R³⁰ is a linear C2 to C6 alkyl group;
R^{3′} is H or a cationic entity preferably selected from the group comprising:
metal cations advantageously selected from the subgroup comprising sodium, potassium, calcium and magnesium,
organic cations advantageously selected from the subgroup comprising:
cations based on amine,
cations based on oligoamine,
cations based on polyamine (polyethylenimine being particularly preferred),
cations based on amino acid(s) advantageously selected from the class comprising cations based on lysine or arginine,
and cationic polyamino acids advantageously selected from the subgroup comprising polylysine and oligolysine;
R⁵⁰ is a C2 to C6 alkyl, dialkoxy or diamine group;
R⁴ is a direct bond or a “spacer” based on 1 to 4 amino acid units;
A independently is a radical -CH₂-(aspartic unit) or -CH₂—CH₂-(glutamic unit);
n′+m′ or n″ is defined as the degree of polymerization and varies from 10 to 1000 and preferably between 50 and 300.

9. Formulation according to claim 7 or 8, characterized in that the n HG of the PO each independently of one another are a monovalent radical of the formula below: 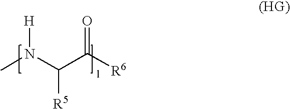
in which:
R⁵ is a methyl (alanine), isopropyl (valine), isobutyl (leucine), sec-butyl (isoleucine) or benzyl (phenylalanine);
R⁶ is a hydrophobic radical containing from 6 to 30 carbon atoms;
1 varies from 0 to 6.

10. Formulation according to claim 9, characterized in that all or some of the hydrophobic radicals R⁶ of the PO are independently selected from the group of radicals comprising:
a linear or branched alkoxy containing from 6 to 30 carbon atoms and optionally containing at least one heteroatom (preferably O and/or N and/or S) and/or at least one unit of unsaturation,
an alkoxy containing 6 to 30 carbon atoms, having one or more fused carbocyclic rings and optionally containing at least one unit of unsaturation and/or at least one heteroatom (preferably O and/or N and/or S),
an alkoxyaryl or an aryloxyalkyl having 7 to 30 carbon atoms and optionally containing at least one unit of unsaturation and/or at least one heteroatom (preferably O and/or N and/or S).

11. Formulation according to claim 9 or 10, characterized in that the hydrophobic radical R⁶ of the graft of the PO is derived from an alcohol precursor selected from the group comprising octanol, dodecanol, tetradecanol, hexadecanol, octadecanol, oleyl alcohol, tocopherol and cholesterol.

12. Formulation according to claim 6, characterized in that the PO consists of an alpha-L-glutamate or alpha-L-glutamic homopolymer.

13. Formulation according to claim 6, characterized in that the PO consists of an alpha-L-aspartate or alpha-L-aspartic homopolymer.

14. Formulation according to claim 6, characterized in that the PO consists of an alpha-L-aspartate/alpha-L-glutamate or alpha-L-aspartic/alpha-L-glutamic copolymer.

15. Formulation according to claim 14, characterized in that, in the PO, the distribution of the aspartic and/or glutamic units carrying grafts containing at least one HG unit is such that the resulting polymer is either random or of the block type or of the multiblock type.

16. Formulation according to claim 1, characterized in that the molecular weight of the PO is between 2000 and 100,000 g/mol and preferably between 5000 and 40,000 g/mol.

17. Formulation according to claim 7, characterized in that the hydrophobic radical R⁶ of the graft of the PO is derived from an alcohol precursor formed of tocopherol, and in that:
1%≦[n/(n+m)]×100≦10%,
preferably 3.5%≦[n/(n+m)]×100≦7.5%,
n+m varies from 100 to 400 and preferably between 120 and 300.

18. Formulation according to claim 7, characterized in that the hydrophobic radical R⁶ of the graft of the PO is derived from an alcohol precursor formed of cholesterol:
1%≦[n/(n+m)]×100≦10%,
preferably 3.5%≦[n/(n+m)]×100≦6.5%,
n+m varies from 100 to 400 and preferably between 120 and 300.

19. Formulation according to claim 17 or 18, characterized in that the concentration of polymer [PO] is between 15 and 50 mg/ml.

20. Formulation according to any one of claims 1 to 19, characterized in that its viscosity is less than or equal to 5 Pa.s at 25° C.

21. Formulation according to any one of claims 1 to 20, characterized in that the hydrophobically modified polymers PO are selected from the group comprising polyamino acids, polysaccharides (preferably those in the subgroup comprising pullulans and/or chitosans and/or mucopolysaccharides), gelatins and mixtures thereof.

22. Formulation according to any one of claims 1 to 21, characterized in that its weight fraction of interleukin(s) not associated with the submicronic particles [non-associated interleukin(s)], in %, is such that:
[non-associated interleukin(s)]≦1,
preferably [non-associated interleukin(s)]≦0.5,
most preferably [non-associated interleukin(s)]≦0.1.

23. Formulation according to any one of claims 1 to 22, characterized in that the interleukin is interleukin 2.

24. Formulation according to any one of claims 1 to 23, characterized in that the additional active principle(s) other than the interleukin(s) is (are) a protein, a glycoprotein, a protein bonded to one or more polyalkylene glycol chains [preferably polyethylene glycol (PEG) chains: “PEGylated protein”], a polysaccharide, a liposaccharide, an oligonucleotide, a polynucleotide or a peptide, this (these) additional active principle(s) preferably being selected from haemoglobins, cytochromes, albumins, interferons, cytokines, antigens, antibodies, erythropoietin, insulin, growth hormones, factors VIII and IX, haemopoiesis stimulating factors, and mixtures thereof.

25. Formulation according to any one of claims 1 to 24, characterized in that it is injectable by the parenteral, subcutaneous, intramuscular, intradermal, intraperitoneal or intracerebral route or into a tumour.

26. Formulation according to any one of claims 1 to 25, characterized in that it is intended for the preparation of drugs, particularly for administration by the parenteral, subcutaneous, intramuscular, intradermal, intraperitoneal or intracerebral route or into a tumour, or by the oral, nasal, vaginal or ocular route.

27. Process for the preparation of drugs, particularly for administration by the parenteral, subcutaneous, intramuscular, intradermal, intraperitoneal or intracerebral route or into a tumour, or by the oral, nasal, vaginal or ocular route, characterized in that it consists essentially in using at least one formulation according to any one of claims 1 to 26.

28. Derived product, characterized in that it comprises submicronic particles formed of non-covalent PO/AP associations as defined in claim 1, and in that it is obtained from the formulation according to any one of claims 1 to 26.

29. Derived product according to claim 28, characterized in that it consists of a powder or a gel.

30. Process for the preparation of the formulation according to any one of claims 1 to 26, characterized in that it consists essentially in:
taking a colloidal suspension of nanoparticles of at least one PO,
mixing this colloidal suspension of nanoparticles of PO with at least one interleukin (and one or more other possible active principles), preferably in aqueous solution,
optionally adding at least one excipient,
adjusting the pH and/or the osmolarity if necessary, and
optionally filtering the resulting suspension.

31. Process according to claim 30, characterized in that the AP is (are) in the form of an aqueous suspension or solution for mixing with the colloidal suspension of nanoparticles of PO.

32. Process for the preparation of the formulation according to any one of claims 1 to 26, characterized in that it consists essentially in:
taking a powder of at least one polymer PO,
mixing this powder with an aqueous suspension or solution of at least one interleukin (and one or more other possible active principles), preferably in aqueous solution,
optionally adding at least one excipient,
adjusting the pH and/or the osmolarity if necessary, and
optionally filtering the resulting suspension.

33. Process for the preparation of the formulation according to any one of claims 1 to 26, characterized in that it consists essentially in:
taking a powder produced by drying the liquid formulation according to any one of claims 1 to 26,
mixing this powder with an aqueous liquid medium, preferably with stirring,
optionally adding at least one excipient,
adjusting the pH and/or the osmolarity if necessary, and
optionally filtering the resulting suspension.

34. Process for the preparation of a powder derived from the formulation according to any one of claims 1 to 26, characterized in that said powder is obtained by drying the formulation according to any one of claims 1 to 26.
